(19) United States
(12) Patent Application Publication
(10) Pub. No.: US 2003/0135332 A1
(43) Pub. Date: Jul. 17, 2003

(54) Circuit and method for pipelined code sequence searching

(75) Inventor: J. Barry Shackleford, Portola Valley, CA (US)

Correspondence Address: HEWLETT-PACKARD COMPANY, Intellectual Property Administration, P.O. Box 272400, Fort Collins, CO, 80527-2400, US

(73) Assignee: Hewlett Packard Company

(21) Appl. No.: 10/047,045

(22) Filed: Jan. 14, 2002

(51) Int. Cl. G01V 3/00; G01V 7/00; G06F 19/00; G01N 33/48; G01N 33/50; G06F 7/50; H03K 19/21

(52) U.S. Cl. 702/20; 326/53

(57) ABSTRACT

A method and apparatus for searching a parent code sequence for a target code sequence. In various embodiments, a first circuit arrangement selects and stores subsets of codes of the parent code sequence. A matching circuit determines in parallel matches between the subset of codes and the target code sequence, and provides a programmed binary value for each match. The binary values provided by the matching circuit are summed in a pipelined fashion.

### FIELD OF THE INVENTION

The present invention generally relates to pattern matching, and more particularly to searching a parent code sequence for a target code sequence.

### BACKGROUND

A genome is a double-stranded DNA molecule held together by pairs of chemical components called nucleotides. There are only four nucleotide types composed of the bases adenine, thymine, guanine, and cytosine, often abbreviated as A, T, G, and C respectively, and hereinafter referred to as “characters.” The structure of the genome can be described therefore, as a long sequence of nucleotides. A human genome includes for example, a sequence of approximately 3 billion nucleotides. The sequence of nucleotides can be represented in a database as a sequence of characters. In the laboratory, a researcher will derive a particular sequence of several hundred nucleotides (i.e., a sequence of several hundred characters, each character representative of a nucleotide) through experimentation, and be interested in where the particular character sequence occurs in a genome (human or otherwise).

Genetic information for a number of organisms has been catalogued in relational computer databases, recently including the human genome. However, many of the databases used in biological and medical research are depository, i.e., sequences may be entered multiple times from different sources. Depository databases are not edited for accuracy, and contain sequence mistakes. Many mistakes are present when the sequences are entered, and remain in the databases until removed or corrected by the source of the sequence information. One database may contain multiple versions of the same gene sequence where the gene sequences have minor variations from one another, each version having different sources and names. Generally, depository databases do not provide any means by which to identify the correct sequence. Complications in analyzing genetic information arise from errors not only in the genome database (e.g., the searched character sequence), but also in the experimentally-determined data (e.g., the target character sequence). Sequence errors typically take the form of “spelling errors,” and spurious insertions or deletions of characters. In illustration of a “spelling error” for example, the erroneous character sequence “CATGAG” occurs where a “G” character is erroneously substituted for a “T” character instead of the correct character sequence “CATTAG.” In an example of a spurious character inclusion, the erroneous character sequence “CATTTAG” includes an additional “T” character, as compared to the correct character sequence “CATTAG.” A spurious character deletion is illustrated by the erroneous character sequence “CATAG” which is missing a “T” character, as compared to the correct character sequence “CATTAG.”

In genome projects, an organism's genome is studied to determine the sequence and placement of its genes and their relationship to other sequences and genes within the genome or to genes in other organisms. Methods to search genetic databases quickly, to analyze nucleic acid sequence information, and to predict protein sequence, structure and function from DNA sequence data can be very powerful in determining the relatedness of certain gene sequences with respect to other gene sequences.

Conventional computerized tools for analyzing character pattern information are primarily targeted towards performing rote comparative procedures between character sequences. Errors in the target sequence and/or the database data can lead to sequence over-matching (i.e., matches including at least one erroneous sequence), as well as sequence under-matching (i.e., failure to detect actual sequence matches due to errors in the target and/or searched sequences).

A system and method that address the aforementioned problems, as well as other related problems, are therefore desirable.

### SUMMARY OF THE INVENTION

In various embodiments, the invention provides a method and apparatus for searching a parent code sequence for a target code sequence. In one example embodiment, a circuit arrangement for searching a parent code sequence for a target code sequence includes a circuit arrangement for selecting and storing subsets of codes of the parent code sequence. A matching circuit determines in parallel matches between the subset of codes and the target code sequence, and provides a programmed binary value for each match. The binary values provided by the matching circuit are summed in a pipelined fashion.

Various example embodiments are set forth in the Detailed Description and Claims which follow.

### BRIEF DESCRIPTION OF THE DRAWINGS

Various aspects and advantages of the invention will become apparent upon review of the following detailed description and upon reference to the drawings in which:

FIG. 1 is a circuit arrangement for locating sequence of characters within a larger sequence of characters, according to an example embodiment of the present invention.

FIG. 2 is a circuit arrangement for locating a sequence of nucleotide characters within a genome, according to another embodiment of the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION

Various embodiments of the present invention are described in terms of a circuit arrangement for searching a parent character sequence representative of a human genome for a target gene character sequence. Those skilled in the art will appreciate, however, that the invention could be implemented using other circuit elements and/or adapted to other applications that involve searching for a particular sequence of codes within a much larger sequence of codes. Thus, the invention is not limited to codes that represent character data, nor to applications where there are only a small number of codes to match.

In one example embodiment, a circuit arrangement for searching a parent code sequence for a target code sequence includes a shift register arrangement having a plurality of stages for selecting and storing subsets of codes of the parent code sequence, each stage storing a code of the subset of codes. According to one example implementation, each subset of codes includes n contiguous codes from the parent code sequence. The shift register arrangement is adapted to periodically shift the subset of codes to form a new subset of codes with another code from the parent code sequence in a leading stage. A matching circuit coupled to the shift register arrangement ascertains code position matches between the subset of codes in the stages of the shift register arrangement and codes in the corresponding code positions of the target code sequence, and provides a programmed binary value for each code position match. The binary values provided by the matching circuit are summed in a pipelined adder arrangement coupled to the matching circuit.

According to one aspect of the present invention, a probability of being the target code sequence is determined for each respective subset of codes as the sum of the binary values for code position matches for the respective subset of codes divided a total quantity of code positions in the target code sequence. The probability for each respective subset of codes is associated with a unique identifier representative of a location within the parent code sequence at which the respective subset of codes exists, thereby “locating” the subsets of the parent code sequence likely to be the target code sequence.

In one example application, the pipelined adder arrangement is a pipelined adder tree. According to another example application, the pipelined adder arrangement includes at least one stage of pipelined carry-save adders coupled to at least one stage of pipelined carry-propagate adders. Each stage of the pipelined carry-save adders is adapted to provide a plurality of binary vectors responsive to the quantity of code position matches, and each stage of the pipelined carry-propagate adders is adapted to add the plurality of binary vectors.

In a further example application, a pipelined summing circuit is coupled to the pipelined adder arrangement, and adapted to determine a moving sum of code position matches for a plurality of subsets of codes. According to one example implementation, the plurality of subsets of codes includes at least a most recent subset of codes and a next most recent subset of codes. According to another example implementation, the plurality of subsets of codes includes at least a first subset of codes and a prior subset of codes, an intervening subset of codes being processed (but not included in the moving sum) between the first subset of codes and the prior subset of codes.

According to one example implementation, each stage of the shift register arrangement is adapted for storage of a code of character data. According to another example implementation, each stage of the shift register arrangement is adapted for storage of a code of a plurality of character data.

In one example implementation, the matching circuit includes a plurality of programmable lookup tables. Each lookup table has an input terminal coupled to an output terminal of a corresponding stage of the shift register arrangement, and each lookup table is configured to provide a programmed value responsive to an input code value. For example, the lookup tables are each respectively addressed with the code stored in an associated stage of the shift register arrangement, and configured to generate respective signals of the programmed binary value when addressed by codes equal to codes of the target code sequence. In this manner, the lookup tables generate respective signals of the programmed binary value when the code stored in a particular stage of the shift register arrangement matches a corresponding code position of the target code sequence.

In one example application, the parent code sequence is representative of a genome, for example a human genome. Each code of the parent code sequence being representative of a nucleotide type (e.g., adenine, thymine, guanine and cytosine).

In another example embodiment, an apparatus for searching a parent code sequence for a target code sequence includes a first circuit arrangement for periodically selecting subsets of codes of the parent code sequence. Each code in the parent code sequence having a parent-relative position, and each code in the subset having a relative subset-code position defined by the parent-relative position. Furthermore, each subset of codes differs from other subsets by parent-relative positions of the codes in the subset. A second circuit arrangement is coupled to the first circuit arrangement and adapted to determine in parallel whether each code at a subset-code position in the subset of codes is equal to a code of the target code sequence in a corresponding target-code position. The second circuit arrangement is further adapted to generate in parallel signals of a selected binary value for each equality of a subset code and the target code. A summing arrangement, coupled to the second circuit arrangement, is adapted to sum the signals of the selected binary values.

The remainder of this description is drafted with reference to a particular application involving character matching. It will be appreciated, however, that the invention is not limited to matching codes that represent character data. The invention is applicable to matching a sequence of codes without being limited to the type of information represented by the codes.

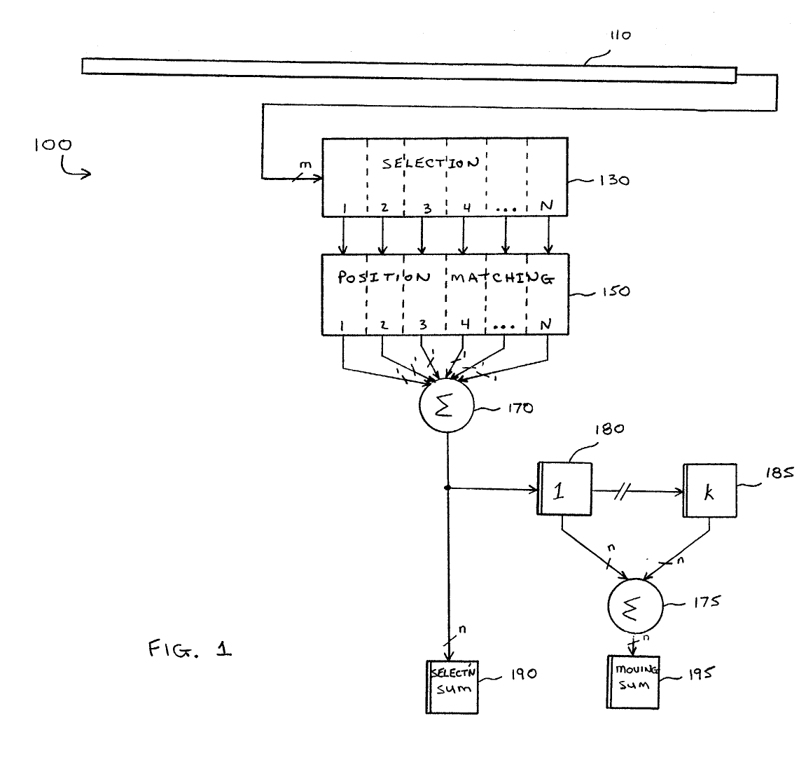

FIG. 1 illustrates a circuit arrangement 100 for searching a parent character sequence for a target character sequence, according to an example embodiment of the present invention. The parent character sequence is contained in a sequence database, which is stored in memory element 110. In an example implementation, the sequence database is representative of a genome, each character of the parent character sequence representative of one of the four nucleotide types. According to a more particular implementation, the parent character sequence is representative of a human genome.

Memory element 110 is communicatively coupled to a selection circuit 130. Selection circuit 130 is adapted to receive a serial stream of characters from memory element 110. Selection circuit 130 includes a plurality of stages, for example N stages where N corresponds to the quantity of characters in the target character sequence. Characters of the parent character sequence are sequentially clocked in series from the memory element through the stages of the selection circuit 130 according to a periodic signal (e.g., a clock signal, not shown). Stages of the selection circuit temporarily hold at least one character. A character held in one particular stage of the selection circuit is subsequently clocked into the next (i.e., adjacent) stage according to the periodic signal, until the character finally exits the selection circuit at the Nth stage.

A position matching circuit 150 is coupled to the selection circuit. According to one example implementation, each stage of the selection circuit is coupled to a respective section of the position matching circuit. Each section of the position matching circuit is adapted to determine whether the character in the corresponding stage of the selection circuit matches a selected character in a particular position of the target character sequence. For example, the first section of the position matching circuit is adapted to determine whether the character in the first stage of the selection circuit matches a the first character of the target character sequence.

Each section of the position matching circuit matches against one or more characters in one example implementation. The set of characters in each section are defined by the characters of interest at each character position in the target character sequence. For example, to search for a character sequence of ACGT or CAGT, the character set for the first character position would include A and C. The first section of the position matching circuit identifies a character position match if the character clocked into the first stage of the selection circuit is either an A or a C. The character set for the third character position would include only a single character, G.

A pipelined counting circuit 170 is coupled to the matching circuit 150, the counting circuit being adapted to sum the quantity of matching character positions. In one example implementation, counting circuit 170 is a pipelined adder tree. The speed of each step of the pipelined counting circuit equals the speed of the character position matching function. Counting circuit 170 is pipelined, the sum of character position matches being computed, after some initial processing delay, at the same rate that characters are clocked into the selection circuit. The sum of character position matches, determined by the counting circuit 170, is presented at a selection sum register.

Those skilled in the relevant art will appreciate that a portion of the parent character sequence which matches at each and every character position (i.e., stage of the selection circuit, and section of the matching circuit) is indicative of a character sequence within the parent character sequence that matches exactly the target character sequence. For example, if the target character sequence is six character long, then a sum of six character position matches is indicative of an exact match between the selected character sequence (i.e., a portion of the parent character sequence) and the target character sequence.

Errors can occur, however, within the parent character sequence and/or within the target character sequence. Therefore, complete (100%) matching may not occur at every character position between the target character sequence and the selected character sequence. For example a spelling error (i.e., a character having an incorrect value or identity) in either the parent character sequence, or the target character sequence, will skew character position match sum that are indicative of character sequences of interest.

In illustration of a spelling error, the erroneous character sequence “CATGAG” occurs where a “G” character is erroneously substituted for a “T” character instead of the correct character sequence “CATTAG.” Desiring to search for the character sequence “CATTAG,” a sum of 5 (of 6, or 83%) results when the sequence “CATGAG” containing the misspelling is selected from the parent sequence. The misspelling camouflages the extent of character position matching. Conversely, if the error occurs in the target character sequence, searching for “CATGAG” would also result in a sum of 5 (of 6, or 83%) when the correctly-spelled sequence “CATGAG” of interest is selected by the selection circuit. Selecting a sequence “CATGAG” would result in a sum of 6 (of 6, or 100%); however, unless the selected character sequence also includes the same spelling error, the selected character sequence is not actually of interest. Generally, the greater the sum of character position matches with respect to the total number of characters in the target character sequence, the greater the likelihood of a “match.” In this manner, the circuit of the present invention indicates matches between the target character sequence and a selected character sequence even in the presence of misspelling errors.

According to one aspect of the present invention, a locator circuit (not shown) receives each sum determined by the counting circuit 170. The locator circuit tracks the location of the selected character sequence selected within the parent character sequence. In one example implementation, the locator circuit is a simple counter which indicates the position within the parent character sequence of the most-recent character clocked into the first stage of the selection circuit. The locator circuit counter is decremented as each new sequential character is clocked into the selection circuit from the memory element. Other known methods for associating a location of the selected character sequence within the parent character sequence are contemplated.

The parent character sequence and/or target character sequence may include errors in the form of spurious insertions or deletions. Spurious insertions or deletions of k characters are detected by the circuit of the present invention by computing a moving sum. As a character sequence that includes a spurious character insertion is clocked into and through the selection circuit 130, at some point the characters on one side of the spurious character insertion will match a portion of the target character sequence. Subsequently (e.g., after k more characters are clocked into the selection circuit), characters on the other side of the spurious character insertion will match the target character sequence. Together, the total of the character position matches before and after the spurious character insertion will be 100% (e.g., 6 of 6, or 5 of 5; assuming no additional spelling type errors). Therefore, a “moving sum” equal to the two sums generated k characters apart is indicative of a “match” between the target character sequence and the selected character sequence in the presence of an character insertion error of length k.

Referring again to FIG. 1, the most recent sum out of the pipelined counting circuit 170 is stored in storage element 180, for example a register. The sum out of the pipelined counting circuit 170 k sums ago is stored in a kth storage element 185. The most recent sum and the sum in the kth storage element are added together by the summing circuit 175, and the moving sum result is presented at register 195. For example, to monitor for “matches” between the target character sequence and the selected character sequence including a single character spurious insertion, k is one. Therefore, the most recent character position matches sum is added to the previous sum of character position matches. As the characters from the parent character sequence are being clocked through the selection circuit, at some point the characters on one side of the spurious character will match the target character sequence, but the characters on the other side of the spurious character will likely not match since their position relative to the matching characters is shifted by the presence of the spurious character. The sum generated for the selected character sequence will reflect the matches of characters on the one side of the spurious character. At the next cycle, the characters in the selection circuit are shifted over one stage, and the characters on the other side of the spuriously inserted character will align (and thus, match-up with) the corresponding characters of the target character sequence. The sum corresponding to this selected character sequence will include those character position matches on the other side of the spuriously inserted character. Adding the present sum with the previous sum will generate a moving sum totaling at least 100% of the number of characters in the target character sequence.

According to one aspect of the present invention, the moving sum is computed from a pipelined summing circuit (e.g., devices 180, 185, 175, and 195) so that the moving sums are generated at a rate equal to the rate at which characters are clocked into the selection circuit.

Monitoring of “matches” between the target character sequence and the selected character sequence in the presence of spurious insertions of more than one character can be similarly implemented, for example where k equals two. Monitoring for “matches” between the target character sequence and the selected character sequence in the presence of k spurious deletions is similarly accomplished; the location of the “matching” character sequence being offset by k characters.

In one embodiment, circuit arrangement 100 is implemented in a programmable logic device, such as a field programmable gate array. It will be appreciated, however, that other types of devices, for example, and ASIC, may be more suitable depending on system requirements.

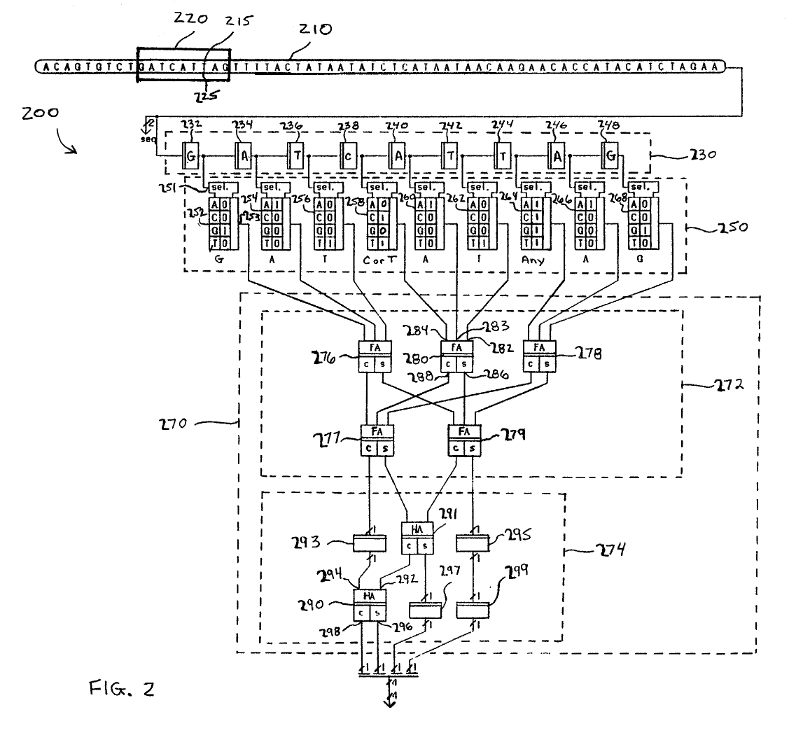

FIG. 2 illustrates a circuit arrangement 200 for searching a parent character sequence for a target character sequence, according to another embodiment of the invention. The parent character sequence is contained in a sequence database, which is stored in memory element 210. In one implementation, the sequence database is representative of a genome, each character of the parent character sequence representative of one of the four nucleotide types. According to a more particular implementation, the parent character sequence is representative of a human genome. Window 220 indicates a subset of the parent character sequence, which may be for example a test character sequence selected from the parent character sequence.

Each character of the parent character sequence occupies a character position within the parent character sequence, for example a “T” character is located at character position 215 (the value 215 is a reference number and not indicative of a relative or absolute position) within the parent character sequence. Similarly, each character of the subset character sequence occupies a character position within the subset character sequence, for example a “T” character is located at character position 225 (the value 225 is a reference number and not indicative of a relative or absolute position) within the subset character sequence. As is observable from FIG. 2, character position 215 within parent character sequence corresponds to character position 225 within the subset character sequence delineated by window 220.

Memory element 210 is communicatively coupled to a selection circuit, for example a pipeline register arrangement 230 in one example embodiment. Pipeline register arrangement 230 is adapted to receive a characters from memory element 210. Register 230 includes a plurality of stages, for example a first stage 232, a second stage 234, a third stage 236, a forth stage 238, a fifth stage 240, a sixth stage 242, a seventh stage 244, an eighth stage 246, and a ninth stage 248 as shown. In further implementations, register 230 is configured to include a stage for each character of a target character sequence, for example hundreds, or thousands, or more stages.

Characters of the parent character sequence are read from the memory element and shifted through the pipeline register arrangement according to a periodic signal (e.g., a clock signal, not shown). Each stage of the pipeline register arrangement temporarily holds a single character, for example FIG. 2 illustrates stage 236 holding a “T” character corresponding to character position 215 of the parent character sequence. A character held in one particular stage of the sequential pipeline register is subsequently clocked into the next (i.e., adjacent) stage in the sequential pipeline register according to the periodic signal. For example, character “T” was clocked into stage 236 from stage 234, and is next clocked to stage 238, and subsequently into stage 240, and so on until character “T” is finally clocked out of the sequential pipeline register from stage 248.

By the above-described mechanism, the pipeline register arrangement holds a subset of contiguous characters of the parent character sequence at time t, for example, FIG. 2 illustrates the pipeline sequence register holding the subset indicated by window 220. At time t+1, character “G” in stage 148 is clocked out of the sequential pipeline register. All other characters are simultaneously clocked to the right one stage, and a “T” character from memory element 210 is clocked into stage 232. By this operation, the pipeline register arrangement holds the next subset of contiguous characters of the parent character sequence, effectively shifting window 220 one character to the left along the parent character sequence.

A matching circuit 250 is coupled to the pipeline sequence register. Matching circuit 250 includes a plurality of lookup tables (LUTs) in one example embodiment, one lookup table per pipeline sequence register stage. Each respective LUT has a select input terminal and an output terminal. For example, LUT 252 has a select input terminal 251, and an output terminal 253. Each LUT is programmed to output selected binary values responsive to the character value present at its selection input. For example LUT 252 outputs a binary one when the character code at input terminal 251 represents the character “G”, and outputs binary 0 for all other input values. Since each LUT is coupled to a respective pipeline sequence register stage as shown in FIG. 2, each LUT functions to determine whether a character identity at a particular character position of the selected (test) character sequence matches at least one character pre-programmed into the LUT. By pre-programming each of the plurality of LUTs to output a selected binary value when addressed by a character code of the target character sequence, the matching circuit provides output values that indicate the quantity of character positions of the selected character sequence having characters that match the target character sequence.

The target character sequence is pre-programmed into matching circuit 250. In the example implementation illustrated in FIG. 2, each LUT is pre-programmed to determine a match at one character position of the target character sequence. For example, LUT 252 is configured and arranged to determine a match between the first character position of the subset (e.g., test) character sequence and the corresponding first character position of the target character sequence. LUT 252 is pre-programmed to provide a binary “1” output at output terminal 253 when a “G” character is presented at selection input 251, and provide a binary “0” output at output terminal 253 otherwise (e.g., when an A, C, T or other character is presented at select input 251). Similarly, LUT 254 is adapted to determine a match between the second character position of the subset (e.g., test) character sequence and the corresponding second character position of the target character sequence; LUT 256 is adapted to determine a match between the third character position of the subset (e.g., test) character sequence and the corresponding third character position of the target character sequence; LUT 258 is adapted to determine a match between the forth character position of the subset (e.g., test) character sequence and the corresponding forth character position of the target character sequence; LUT 260 is adapted to determine a match between the fifth character position of the subset (e.g., test) character sequence and the corresponding fifth character position of the target character sequence; LUT 262 is adapted to determine a match between the sixth character position of the subset (e.g., test) character sequence and the corresponding sixth character position of the target character sequence; LUT 264 is adapted to determine a match between the seventh character position of the subset (e.g., test) character sequence and the corresponding seventh character position of the target character sequence; LUT 266 is adapted to determine a match between the eighth character position of the subset (e.g., test) character sequence and the corresponding eighth character position of the target character sequence; and LUT 268 is adapted to determine a match between the ninth character position of the subset (e.g., test) character sequence and the corresponding ninth character position of the target character sequence.

As will be understood by those skilled in the relevant art, the plurality of LUTs are programmed to detect in a test character sequence, a particular nucleotide at a specific character position of a target character sequence (e.g., a nucleotide at a specific point in a gene sequence of interest), when the sequence of parallel LUTs are respectively pre-programmed with characters of the target character sequence. Below each LUT, is the character(s) for which each respective LUT is programmed to test. Each LUT also shows a left column of selection input character identities (e.g., A, C, G, or T), and a right column of output values corresponding to each of the selection input identities (e.g., one of 0 or 1 respectively). For example, LUT 252 is programmed to output a binary 1 when the input character code represents a “G” character, and output a binary 0 for the other character codes.

LUTs can be programmed to indicate whether the character identity at a particular character position of the test character sequence is one of a plurality of characters. For example, LUT 258 is programmed to output a binary 1 when the input character code represents either the character “C” or “T.” In another example, LUT 264 is programmed to provide a “1” output value regardless of character identity of the character present at its selection input terminal. In effect, LUT functions as a “wildcard” character placeholder. This aspect of the present invention is important when attempting to locate in the parent character sequence, a target character sequence that includes at least one character position for which the character identity is unimportant. In one application, this aspect of the present invention can be used to locate a plurality of target character sequences in proximity to one another.

According to the example embodiment illustrated in FIG. 2, the output terminal of each LUT signals whether the character at a particular character position matches at least one character of a target character sequence programmed into the sequence of LUTs. The character position match signals are subsequently summed to determine to what degree the selected subset character sequence matches the target character sequence programmed into matching circuit 250. The output terminals of each LUT are coupled in parallel to a counting circuit 270. Counting circuit 270 includes at least one stage of pipelined carry-save adders 272, and at least one stage of pipelined carry-propagate adders 274.

The pipelined carry-save adder stage includes a first sub-stage of 3-bit (full) adders, 276, 278, and 280 respectively, and a second sub-stage of full adders, 277 and 279 respectively. The output terminal for each LUT is uniquely coupled to an input terminal of one of the first sub-stage of parallel 3-bit (full) adders (e.g., adders 276, 278 and 280). For example, the output terminal of LUT 258 is coupled to the carry bit input terminal of full adder 280; the output terminal of LUT 260 is coupled to the second bit input terminal of full adder 280; and the output terminal of LUT 262 is coupled to the first bit input terminal of full adder 280. Each adder 276-280, has a sum output terminal (e.g., terminal 286 for adder 280), and a carry output terminal (e.g., terminal 288 for adder 280).

The sum output terminals of the three full adders in the first sub-stage are respectively coupled to input terminals of adder 279. The carry output terminals of the three full adders in the first sub-stage are respectively coupled to input terminals of adder 277. As will be appreciated by those skilled in the relevant art, the pipelined carry-save adder stage provides binary vectors representing the final sum, which are subsequently added together in the pipelined carry-propagate adder stage. In the example implementation illustrated in FIG. 2, one binary vector is provided by adders 277, and a second binary vector is provided by adder 279. Because each sub-stage of the pipelined carry-save adder stage is pipelined, the summation of the character position matches, as provided by the LUTs, occurs at the same periodic clocking speed as the matching circuit (e.g., LUT) operation.

The carry output terminal of adder 279 and the sum output terminal of adder 277 are coupled to input terminals of a 2-bit (half) adder in a first sub-stage portion of the pipelined carry-propagate adder stage. The sum output terminal of adder 279 is coupled to a propagation delay device 295 having a delay equal to the computational time of half adder 291. Similarly, the carry output terminal of adder 277 is coupled to a propagation delay device 293 also having a delay equal to the computational time of half adder 291.

A second sub-stage portion of the pipelined carry-propagate adder stage adds the values at the carry output terminal of half adder 291 to the value of the carry output terminal of adder 277 (via propagation delay device 293). The output terminal of propagation delay device 293 is coupled to one input terminal of half adder 290, and the carry output terminal of half adder 291 is coupled to the other input terminal of half adder 290. The value at the sum output terminal of full adder 279 (via propagation delay device 295) is further delayed by propagation delay device 299 having a delay equivalent to the computational time of half adder 290. The value at the sum output terminal of half adder 291 is also delayed by the computational time of half adder 290 by propagation delay device 297.

The configuration of the counting circuit of the present invention provides a binary number representative of the quantity of character position matches between the target character sequence programmed into the LUTs of the matching circuit, and the subset character sequence selected by the selection circuit and contained in the pipeline sequence register. The output terminal of propagation delay device 299 provides the binary one's place value, the output terminal of propagation delay device 297 provides the binary two's place value, the sum output terminal 296 provides the binary four's place value, and the carry output terminal 298 provides the eight's place value.

The configuration illustrated in FIG. 2 is expandable to target character sequences of greater length. The pipelined counting circuit may require additional depth depending on the length of the target character sequence and thus, the matching circuit length. Additional counting circuit depth will increase latency of the pipeline before a sum is generated, otherwise circuit operation is similar to that described above.

According to another embodiment of the present invention, a parent character sequence is searched using a parallel arrangement of circuits, each circuit being configured as shown in FIG. 2 and arranged to begin searching the parent character sequence at a different location. For example, two searching circuits as described in FIG. 2 are used in one example implementation, the first searching circuit arranged to search from one end of the parent character sequence and the second searching circuit arranged to search from the middle of the parent character sequence. Searching very long parent character sequences in parallel, for example the human genome, increases search speed. Some overlap, equal to the length of the target character sequence is necessary to ensure matching character sequences are not occurring at the point of division of the parent character sequence between parallel searching circuits.

In addition to the example embodiments described above, other aspects and embodiments of the present invention will be apparent to those skilled in the art from consideration of the specification and practice of the invention disclosed herein. It is intended that the specification and illustrated embodiments be considered as examples only, with a true scope and spirit of the invention being indicated by the following claims.

What is claimed is:

1. A circuit arrangement for searching a parent code sequence for a target code sequence, comprising:
a shift register arrangement having a plurality of stages, wherein each stage stores a code of a subset of codes of the parent code sequence, and the shift register arrangement is adapted to periodically shift the subset of codes to form a new subset of codes with another code from the parent code sequence in a leading stage;
a matching circuit coupled to the shift register arrangement, the matching circuit adapted to ascertain code position matches between the subset of codes in the stages of the shift register arrangement and codes in corresponding code positions of the target code sequence, and provide a programmed binary value for each code position match; and
a pipelined adder arrangement coupled to the matching circuit, the adder arrangement adapted to sum the binary values for code position matches for each respective subset of codes.

2. The circuit arrangement of claim 1, wherein each stage of the shift register arrangement is adapted for storage of a code of character data.

3. The circuit arrangement of claim 1, wherein each stage of the shift register arrangement is adapted for storage of a code of a plurality of character data.

4. The circuit arrangement of claim 1, wherein the pipelined adder arrangement is a pipelined adder tree.

5. The circuit arrangement of claim 1, wherein the pipelined adder arrangement includes at least one stage of pipelined carry-save adders coupled to at least one stage of pipelined carry-propagate adders.

6. The circuit arrangement of claim 5, wherein the at least one stage of pipelined carry-save adders are adapted to provide a plurality of binary vectors responsive to the quantity of code position matches, and the at least one stage of pipelined carry-propagate adders are adapted to add the plurality of binary vectors.

7. The circuit arrangement of claim 1, further comprising a pipelined summing circuit coupled to the pipelined adder arrangement and adapted to determine a moving sum of code position matches for a plurality of subsets of codes.

8. The circuit arrangement of claim 7, wherein the plurality of subsets of codes includes at least a most recent subset of codes and a next most recent subset of codes.

9. The circuit arrangement of claim 7, wherein the plurality of subsets of codes includes a first subset of codes and a prior subset of codes, wherein an intervening subset of codes is processed between the first subset of codes and the prior subset of codes.

10. The circuit arrangement of claim 1, wherein each subset of codes includes n contiguous codes from the parent code sequence.

11. The circuit arrangement of claim 1, wherein the matching circuit includes a plurality of programmable lookup tables, each lookup table having an input terminal coupled to an output terminal of a corresponding stage of the shift register arrangement and configured to provide a programmed value responsive to an input code value.

12. A method for searching a parent code sequence for a target code sequence, comprising:
shifting the parent code sequence through a shift register arrangement having a plurality of stages, wherein the shift register arrangement stores a subset of codes of the parent code sequence and each stage stores a code of the subset of codes, and each shift of the subset of codes forms a new subset of codes with another code from the parent code sequence in a leading stage;
determining in parallel whether the codes in the stages of the shift register arrangement are equal to codes of the target code sequence in corresponding code positions, and generating in parallel signals of a programmed binary value for each equality of a subset code and a target code; and
summing the signals of the programmed binary value in a pipelined adder that generates a sum corresponding to each shift of the shift register arrangement.

13. The method of claim 12, further comprising:
determining, for each respective subset of codes, a probability of being the target code sequence as the sum of the binary values for code position matches for the respective subset of codes divided a total quantity of code positions in the target code sequence; and
associating the probability for each respective subset of codes with a unique identifier representative of a location within the parent code sequence at which the respective subset of codes exists.

14. The method of claim 12, wherein the parent code sequence represents a genome.

15. The method of claim 14, wherein each code of the parent code sequence is representative of a nucleotide type.

16. The method of claim 15, wherein the nucleotide type is selected from the group consisting of adenine, thymine, guanine, and cytosine.

17. The method of claim 14, wherein the genome is a human genome.

18. The method of claim 12, further comprising:
configuring a plurality of lookup tables to generate respective signals of the programmed binary value when addressed by codes equal to codes of the target code sequence; and
addressing the lookup tables with the codes of the subset of codes.

19. The method of claim 12, further comprising generating a moving sum, for n subsets of codes, of sums of the signals of the selected binary value.

20. The method of claim 19, wherein the n subsets of codes includes at least a most recent subset of codes and a next most recent subset of codes.

21. The method of claim 19, wherein the n subsets of codes includes a first subset of codes and a prior subset of codes, wherein an intervening subset of codes is processed between the first subset of codes and the prior subset of codes.

22. The method of claim 12, wherein each subset of codes includes m contiguous codes from the parent code sequence.

23. An apparatus for searching a parent code sequence for a target code sequence, each code in the parent code sequence having a parent-relative position, comprising:
means for periodically selecting subsets of codes of the parent code sequence, each code in the subset having a relative subset-code position defined by the parent-relative position, and each subset of codes differing from other subsets by parent-relative positions of the codes in the subset;
means for determining in parallel whether each code at a subset-code position in a subset of codes is equal to a code of the target code sequence in a corresponding target-code position, and generating in parallel signals of a selected binary value for each equality of a subset code and the target code; and
means for summing the signals of the selected binary value.
